(19) United States
(12) Patent Application Publication
(10) Pub. No.: US 2014/0187940 A1
(43) Pub. Date: Jul. 3, 2014

(54) METHOD OF CALCULATING DISPLACEMENT OF SHEAR WAVE, METHOD OF CALCULATING MECHANICAL MODULUS OF BODY, AND SYSTEM USING THE METHODS

(71) Applicant: SAMSUNG ELECTRONICS CO., LTD., Suwon-si (KR)

(72) Inventors: Dong-geon KONG, Hwaseong-si (KR); Ji-young PARK, Yongin-si (KR); Hyoung-ki LEE, Seongnam-si (KR); Ki-wan CHOI, Anyang-si (KR)

(73) Assignee: SAMSUNG ELECTRONICS CO., LTD., Suwon-si (KR)

(21) Appl. No.: 13/955,384

(22) Filed: Jul. 31, 2013

(30) Foreign Application Priority Data
Dec. 28, 2012 (KR) 10-2012-0157335

(51) Int. Cl. A61B 8/08

(52) U.S. Cl. CPC A61B 8/485
USPC 600/438; 600/437

(57) ABSTRACT

A method of calculating a displacement of a shear wave includes inducing a shear wave in a body, obtaining a plurality of propagation frames including propagation information of the shear wave from an echo signal received from the body, determining a reference frame from among the plurality of propagation frames, and calculating a displacement of the shear wave based on the plurality of propagation frames and the reference frame. A shear modulus may be calculated by using a displacement of the shear wave after the displacement of the shear wave is calculated by comparing the reference frame and propagation frames. A mechanical modulus may be obtained by selecting an ultrasound image as a reference image after a shear modulus is calculated.

### CROSS-REFERENCE TO RELATED APPLICATIONS

This application claims the priority benefit of Korean Patent Application No. 10-2012-0157335, filed on Dec. 28, 2012, in the Korean Intellectual Property Office, the disclosure of which is incorporated herein in its entirety by reference for all purposes.

### BACKGROUND

1. Field

The disclosure herein relates to a method of calculating a displacement of a shear wave, a method of calculating a mechanical modulus of a body by using the shear wave, and an apparatus and system by which one or more of the methods may be implemented.

2. Description of the Related Art

Elastography is a technique used in medical diagnosis for measuring the mechanical properties of biological tissues such as elasticity. Generally, elastography is performed in a medical imaging system as an additional feature to existing imaging procedures such as magnetic resonance imaging (MRI) or ultrasound imaging. It is noted that elastography provides doctors with new clinical information to help them to diagnose various diseases.

### SUMMARY

Provided are methods of calculating a displacement of a shear wave, and systems using the same.

Provided are methods of precisely calculating a mechanical modulus of a tissue of a body and systems using the same.

Additional aspects will be set forth in part in the description which follows and, in part, will be apparent from the description, or may be learned by practice of the presented embodiments.

According to an aspect of the present invention, a method of calculating a displacement of a shear wave includes: inducing a shear wave in a subject (object or body); obtaining a plurality of propagation frames including propagation information of the shear wave from an echo signal received from the body; determining a reference frame from among the plurality of propagation frames; and calculating a displacement of the shear wave on the basis of the plurality of propagation frames and the reference frame.

The reference frame may be the last obtained propagation frame from among the plurality of propagation frames.

The reference frame may be a propagation frame obtained after the shear wave passes through an interest area of the body.

The displacement of the shear wave may not include information of physical changes of the body according to the induction of the shear wave.

The shear wave may be induced by applying an ultrasound signal to the body.

The intensity of the ultrasound signal for inducing the shear wave may be greater than that for obtaining the propagation frames.

The displacement of the shear wave may be obtained by applying a cross-correlation scheme to the propagation frames and the reference frame.

The method may further include displaying an image including the displacement of the shear wave.

According to another aspect of the present invention, a method of calculating a mechanical modulus of a body by using a shear wave includes: calculating a displacement of the shear wave induced in the body and calculating a mechanical modulus of the body from the displacement of the shear wave.

The mechanical modulus may be at least one of a shear modulus, stiffness, and viscosity of the body.

The shear modulus may be calculated from a velocity of the shear wave and a density of the body, and the velocity of the shear wave may be calculated from the displacement of the shear wave.

The method may further include displaying an image including the mechanical modulus.

According to still another aspect of the present invention, an apparatus for processing a shear wave includes: a frame obtaining unit which obtains a plurality of propagation frames including propagation information of a shear wave in a body; and a displacement calculating unit which selects a reference frame from among the plurality of propagation frames, and which compares the reference frame and the plurality of propagation frames to calculate a displacement of the shear wave.

The displacement calculating unit may select the last obtained propagation frame from among the plurality of propagation frames as the reference frame.

The displacement calculating unit may select a propagation frame obtained after the shear wave passes through an area of interest in the subject body as the reference frame.

The displacement of the shear wave may not include information of physical changes of the body according to induction of the shear wave.

According to still another aspect of the present invention, a system for processing a shear wave includes: the apparatus for processing the shear wave; and an ultrasound probe for applying an ultrasound signal to the body.

The ultrasound probe may induce the shear wave by applying the ultrasound signal to the body.

The ultrasound probe may apply the ultrasound signal to the body, and receive an echo signal corresponding to the plurality of propagation frames from the body.

According to another aspect of the present invention, a method of determining characteristics of an object includes: generating a shear wave in an object; applying, after the shear wave is generated, an ultrasound signal to the object and obtaining a plurality of propagation frames from an echo signal corresponding to the ultrasound signal; selecting, after the shear wave is generated, a reference frame from among the plurality of propagation frames; and determining a displacement of the shear wave based on a relationship between the plurality of propagation frames and the reference frame.

The method may further include determining a shear modulus of the object using a travel velocity and the calculated displacement of the shear wave including displacement components corresponding to a plurality of axes.

The ultrasound signal applied to the object after the shear wave is generated may have a smaller period than an ultrasound signal used to generate the shear wave.

After the shear wave is generated, characteristics of a plurality of propagation frames may be combined to obtain a reference frame. Alternatively, a propagation frame having a highest signal to noise ratio among the plurality of propagation frames may be selected as the reference frame.

A non-transitory computer readable medium may have recorded thereon a program to execute any one or more of the methods disclosed herein on a computer.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates an exemplary use environment of a shear wave processing system, according to an embodiment of the present disclosure;

FIG. 2 illustrates a shear wave, according to an embodiment of the present disclosure;

FIG. 3 illustrates an example of an ultrasound probe applying an ultrasound signal to an area of interest.

FIG. 4 illustrates a detailed block diagram of the shear wave processing apparatus of FIG. 1.

FIG. 5 is a flow chart illustrating a method of calculating a mechanical modulus of tissue in a subject body using a shear wave, according to an embodiment of the present disclosure;

FIG. 6 illustrates a reference drawing for explaining shear wave induction and frame obtaining with reference to a period of time;

FIG. 7 illustrates a result of a comparative example of a shear wave displacement with reference to a period of time;

FIG. 8 illustrates a result of an embodiment of a shear wave displacement with reference to a period of time;

FIG. 9 illustrates an image including a shear modulus, according to the comparison result; and

FIG. 10 illustrates an image including a shear modulus, according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

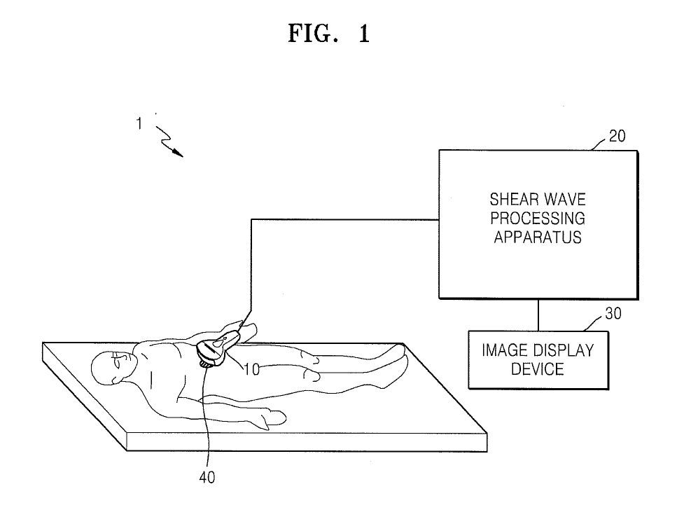

FIG. 1 illustrates an exemplary use environment of a shear wave processing system 1 according to an embodiment of the present disclosure.

Referring to FIG. 1, the shear wave processing system 1 may include an ultrasound probe 10, a shear wave processing apparatus 20, and an image display apparatus (or device) 30. FIG. 1 shows only components of the shear wave processing system 1 according to the present embodiment. However, it will be understood by one of ordinary skill in the art that general-purpose components other than those shown in FIG. 1 may be further included in the shear wave processing system 1. It will also be understood by one of ordinary skill in the art that the ultrasound probe 10, shear wave processing apparatus 20, and/or image display apparatus 30 may be connected with one another over a wired or wireless network, or a combination thereof. Further, components not shown in FIG. 1 may be connected to one or more components of the shear wave processing system 1 shown in FIG. 1 over a wired or wireless network, or a combination thereof.

The ultrasound probe 10 may apply an ultrasound signal to an object or a body (e.g., a patient) in order to induce a shear wave therein, or to obtain an ultrasound image therefrom. The shear wave processing apparatus 20 calculates a shear wave displacement by measuring a shear wave propagating in the body, and calculates a mechanical modulus of a tissue of the body by using the shear wave displacement. In addition, the image display device may display an ultrasound image received from the shear wave processing apparatus 20. The ultrasound image may include a shear wave displacement, and/or a mechanical modulus of the tissue. The image display apparatus 30 displays an ultrasound image created in the shear wave processing apparatus 20. For example, the image display apparatus 30 may include an output device such as a display panel, a liquid crystal display (LCD), organic light emitting diode display (OLED), plasma display panel (PDP), or cathode ray tube (CRT), and the like, for example, or a monitor provided in the shear wave processing system 1.

The shear wave processing system 1 according to the present embodiment may distinguish a normal tissue from an abnormal tissue by calculating the mechanical modulus of the tissue based on ultrasound elastography. The shear wave processing system 1 may be used to determine a tissue state in the body, for example, whether there is an abnormal tissue such as a cancer tumor, or whether treatment of a tissue is completed by calculating the elasticity of the tissue by using ultrasound such as high intensity focused ultrasound (HIFU), etc.

For example, an abnormal tissue has a different stiffness (or density) from a normal tissue, and thus, the existence of an abnormal tissue may be determined based on the difference in stiffness. In detail, an abnormal tissue such as a cancer or a tumor may have higher elasticity than a normal tissue. Due to this, an abnormal tissue such as a cancerous tissue or a tumor has a higher modulus than a surrounding normal tissue. In addition, when necrosis of a tissue occurs by using an ultrasound treatment such as HIFU, the elasticity of the tissue increases as necrosis of the tissue takes place. That is, a change of the tissue state may be represented as a change of the elasticity of the tissue. Therefore, by using an ultrasound signal to obtaining the tissue elasticity, a user may monitor the tissue state in a non-invasive manner even though the user can directly see the tissue inside the body.

The shear wave processing system 1 may be used for disease diagnosis, establishment of a treatment plan, evaluation of treatment progression, etc., by providing a calculation result of a mechanical modulus of the tissue by using the ultrasound image.

For example, before the tissue elasticity is calculated, the ultrasound probe 10 may collect ultrasound waves in a focus area and induce a shear wave. Here, the focus area refers to an area where the ultrasound waves are collected for inducing the shear wave to a body. In order to quantitatively calculate the tissue elasticity by using the ultrasound waves, the ultrasound probe 10 may apply acoustic radiation force impulses (ARFIs) corresponding to the ultrasound waves to the inside of the body. Also, a tissue displacement may be generated when the shear wave is induced in the body by applying the ARFIs.

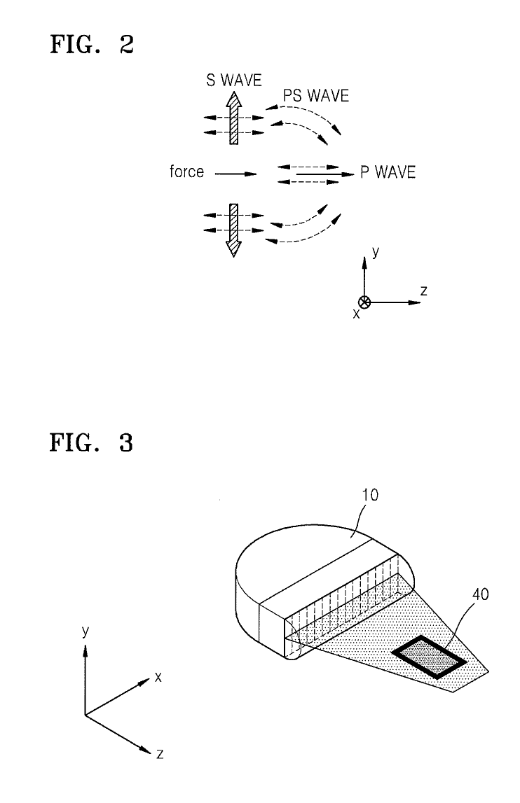

FIG. 2 illustrates a shear wave according to an embodiment of the present disclosure. Referring to FIG. 2, when a point force impulse is applied in a Z axis direction, a p wave, which is a longitudinal wave, an s wave, which is a transversal wave (which may be perpendicular to the p wave), and a ps wave, which is a combined wave of the p and s waves, are generated. Here, the shear wave may refer to an s wave oscillating in a wave traveling direction with respect to a focus area where the force is applied, and traveling in a Y-axis direction.

The point force impulse for generating the shear wave may be applied as an ultrasound signal generated by the ultrasound probe 10, but the present invention is not limited thereto. The shear wave may also be generated by using a HIFU device placed outside the shear wave processing system 1 or a vibrator of an MRI device. That is, one of ordinary skill in the art will understand that the shear wave may be generated in the body by one or more different devices specifically disclosed herein, but that the disclosure is not limited to a single device or those devices specifically disclosed herein which are merely provided by way of example.

Referring back to FIG. 1, the ultrasound probe 10 applies an ultrasound signal to an area of interest. Then the ultrasound probe 10 receives an echo signal reflected by the area of interest. The area of interest may be set such that an amplitude of the generated shear wave may be maintained to have a predetermined value or higher than the predetermined value. For example, the generated shear wave may be desired to have a signal to noise ratio equal to or greater than a predetermined threshold value. For example, the area of interest may be a circle with a diameter of 2 cm, a square, a polygon, or any other geometric shape, etc. In addition, the area of interest may automatically be set by the shear wave processing device 20 without a user's interference in consideration of the amplitude of the generated shear wave, or may be directly set by a user.

FIG. 3 illustrates an example where the ultrasound probe 10 applies ultrasound waves to the area of interest 40.

Referring to FIG. 3, the ultrasound probe 10 may include a one-dimensional array comprising a plurality of transducers, but is not limited thereto. The ultrasound probe 10 may also include a n-dimensional array (e.g., a two-dimensional array) formed of a plurality of transducers. Each transducer may be an element of the ultrasound probe 10 and converts an ultrasound signal into an electric signal. For example, the transducer may be a piezoelectric microband ultrasonic transducer (pMUT) which converts an ultrasound signal into an electric signal through a vibration, a capacitive micro-machined ultrasonic transducer (cMUT), a magnetic micro-machined ultrasonic transducer (mMUT), an optical ultrasound detector, etc.

The transducer may apply an ultrasound signal to the area of interest 40, and receives an echo signal reflected therefrom. For example, when an ultrasound signal in a range of 2 to 18 MHz is applied from the transducer to the area of interest 40, the ultrasound signal is partially reflected by layers positioned between various pieces of tissue. The transducer generates an electrical signal corresponding to the echo signal and sends the same to the shear wave processing apparatus 20. The electrical signal generated by the transducer may be an analog signal or a digital signal.

In addition, the transducers forming the ultrasound probe 10 may form an aperture or a sub-array. For example, the aperture may be a circular shape, rectangular shape, and the like. The aperture indicates some of the transducers forming the ultrasound probe 10. The number of the transducers is not limited thereto. A single transducer may be the aperture.

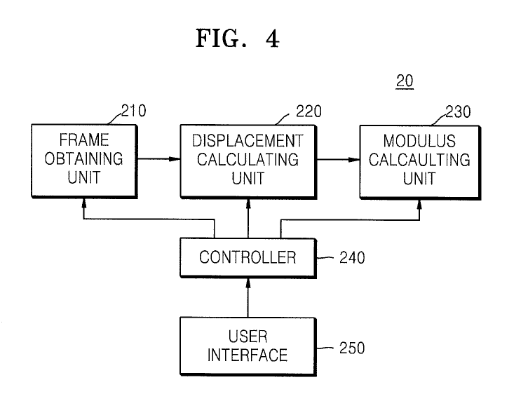

FIG. 4 is a detailed block diagram of the shear wave processing apparatus 20. As shown in FIG. 4, the shear wave processing apparatus 20 may include a frame obtaining unit 210 to obtain a frame including information on propagation of the shear wave, a displacement calculating unit 220 to calculate a displacement of the shear wave, a modulus calculating unit 230 to calculate a mechanical modulus of the body, a user interface 250 to receive instructions from a user, and a controller 240 to control general operations of the shear wave processing apparatus 20 including the frame obtaining unit 210, the displacement calculating unit 220, the modulus calculating unit 230, and/or the user interface 250, for example.

The frame obtaining unit 210, the displacement calculating unit 220, and the modulus calculating unit 230 may respectively be embodied by a single processor or a plurality of processors. Each of the processors may be implemented with an array of multiple logic gates, or a combination of a general purpose microprocessor and a memory containing a program to be executed by the microprocessor. Also, one of ordinary skill in the art will understand that the processor may be implemented with other types of hardware.

The frame obtaining unit 210 may receive from the ultrasound probe 10 the electric signal corresponding to the echo signal, and obtain an ultrasound image by performing beamforming on the electric signal. The ultrasound image may include propagation information of the shear wave in the area of interest. Accordingly, the ultrasound image may be referred to as a propagation frame. The propagation frame may include or not include information on the shear wave.

In detail, the frame obtaining unit 210 may sequentially obtain a plurality of propagation frames after the shear wave is induced in the area of interest 40. The frame obtaining unit 210 may perform beamforming on the electrical signal corresponding to the echo signal according to the time when the transducers respectively apply the ultrasound waves, the time when the echo signal arrives at the transducers from the area of interest 30, or a combination thereof.

The displacement calculating unit 220 calculates a displacement of the shear wave on the basis of degrees of delays of the propagation frames. In addition, the displacement calculating unit 220 may create an image including the displacement of the shear wave. The displacement of the shear wave refers to movement information of the shear wave in time (i.e., with respect to a period of time which has elapsed). That is, the calculated displacement of the shear wave may have displacement components along an x-axis, a y axis, or a z-axis in a coordinate space.

In detail, the displacement calculating unit 220 selects any one of the plurality of propagation frames obtained in the frame obtaining unit 210 as a reference frame, and calculates the displacement of the shear wave on the basis of the reference frame and each of the plurality of propagation frames. The reference frame may refer to a criterion frame for calculating the displacement of the shear wave, and may be any one of the propagation frames. For example, the reference frame may be the last obtained frame of the plurality of the propagation frames, or a propagation frame for the area of interest after the shear wave passes therethrough. The displacement calculating unit 220 may calculate the displacement of the shear wave by applying a cross-correlation scheme between the reference frame and each of the propagation frames.

The displacement calculating unit 220 may calculate the displacement of the shear wave precisely by selecting the reference frame among the plurality of the propagation frames. When the ultrasound signal for inducing the shear wave is applied to a focus area of the area of interest, the shear wave is induced, and physical changes of the tissue at the focus area and around the focus area may occur. As a result, the propagation frames include not only information on propagation of the shear wave, but also information according to physical changes of the tissue. When any one of the propagation frames including the information according to physical changes of the tissue is selected as the reference frame, the information according to physical changes of the tissue is canceled out in the calculating process of the displacement of the shear wave.

In order to precisely obtain the displacement of the shear wave, the reference frame may be a propagation frame only including information according to physical changes of the tissue, or a propagation frame including less information according to the propagation of the shear wave. Accordingly, the reference frame may be the last obtained propagation frame among the plurality of propagation frames, or a propagation frame obtained after the propagation frames pass through the area of interest.

The modulus calculating unit 230 calculates a mechanical modulus of the tissue within the area of interest by using the calculated displacement of the shear wave. The modulus calculating unit 230 may create an image including the mechanical modulus of the area of interest. The calculated mechanical information in the present embodiment may include a shear modulus.

For example, the modulus calculating unit 230 may calculate a shear modulus of the tissue in the area of interest 40 by using displacement components respectively corresponding to 2-dimensional coordinate axes (x-axis or y-axis) or 3-dimensional coordinate axes (x-axis, y-axis, and z-axis). Here, the modulus calculating unit 230 may calculate a shear modulus by using a wave equation of the shear wave. Hereinafter operations of the modulus calculating unit 230 will be described by assuming that the displacement of the shear wave calculated in the displacement calculating unit 220 includes the displacement components corresponding to each of the 3-dimensional axes. When the displacement of the shear wave includes the displacement components respectively corresponding to the 2-dimensional axes (for example, displacement components respectively corresponding to x-axis and y-axis), the modulus calculating unit 230 may calculate the shear modulus by calculating a displacement component corresponding to an axis other than the 2-dimensional axes by using the displacement components respectively corresponding to the 2-dimensional axes.

First, the modulus calculating unit 230 calculates a travel velocity of the shear wave by using the displacement components which respectively correspond to the 3-dimensional axes and are included in the calculated displacements of the shear wave.

$$\frac{{\partial }^{2} u}{\partial {t}^{2}} = {C}_{S}^{2} \cdot ( \frac{{\partial }^{2} u}{\partial {x}^{2}} + \frac{{\partial }^{2} u}{\partial {y}^{2}} + \frac{{\partial }^{2} u}{\partial {z}^{2}} ) \qquad ( 1 )$$

Referring to equation 1, u denotes a displacement of the shear wave, and Cs denotes a travel velocity of the shear wave. In the present embodiment, it is exemplified that the modulus calculating unit 230 calculates the travel velocity of the shear wave by using equation 1. However, the present invention is not limited thereto.

Then, the modulus calculating unit 230 calculates a shear modulus of the tissue in the area of interest 40 by using the calculated travel velocity Cs of the shear wave.

G=ρ×C_{S}² (2)

Referring to equation 2, G denotes a shear modulus, and ρ denotes a density of a medium. Previously, the modulus calculating unit 230 calculates the travel velocity Cs of the shear wave by using equation 1 and ρ is a known value. Accordingly, the modulus calculating unit 230 may calculate the shear modulus G by using equation 2. In the present embodiment, it is exemplified that the modulus calculating unit 230 calculates the shear modulus by using equation 2. However, the present invention is not limited thereto.

On the other hand, the modulus calculating unit 230 may calculate the shear modulus G by using equation 3.

$$ρ \frac{{\partial }^{2} {u}_{z}}{\partial {t}^{2}} = G ( x , y , z ) ( \frac{{\partial }^{2} {u}_{z}}{\partial {x}^{2}} + \frac{{\partial }^{2} {u}_{z}}{\partial {y}^{2}} + \frac{{\partial }^{2} {u}_{z}}{\partial {z}^{2}} )$$

$$⇔ G ( x , y , z ) = \frac{ρ \frac{{\partial }^{2} {u}_{z}}{\partial {t}^{2}}}{\frac{{\partial }^{2} {u}_{z}}{\partial {x}^{2}} + \frac{{\partial }^{2} {u}_{z}}{\partial {y}^{2}} + \frac{{\partial }^{2} {u}_{z}}{\partial {z}^{2}}} \qquad ( 3 )$$

That is, the modulus calculating unit 230 may calculate the shear modulus by using equation 3, which is obtained by combining equations 1 and 2.

As described above, the frame obtaining unit 210 obtains an ultrasound image of the propagation frames, and the displacement calculating unit 220 calculates the displacement of the shear wave. Therefore, the modulus calculating unit 230 may calculate a shear modulus in consideration of all of the calculated displacement components.

In addition, the modulus calculating unit 230 may calculate the travel velocity of the shear wave by using equation 3, and a mechanical modulus such as Young's modulus by using the shear modulus. Also, the modulus calculating unit 230 may calculate viscosity through a frequency analysis of equation 3.

The controller 240 may be included in the shear wave processing apparatus 20 and may communicate with and/or control operations of, the frame obtaining unit 210, the displacement calculating unit 220, the modulus calculating unit 230, and/or the user interface 250, for example.

Meanwhile, the user interface 250 may receive instructions from a user in order to perform an operation or receive an input which may be used to perform an operation (e.g., setting of a parameter) in order to calculate a displacement of a shear wave and/or calculate a mechanical modulus of a body. For example, the user interface 250 may include an input device such as a display panel, a mouse, a keyboard, a touch screen, graphical user interface, pedal, footswitch, voice control unit or microphone, or combinations thereof, and a software module for driving them. Alternatively, the user interface 250 may also be integrated with an image display device (for example, a smartphone, tablet, laptop, and the like).

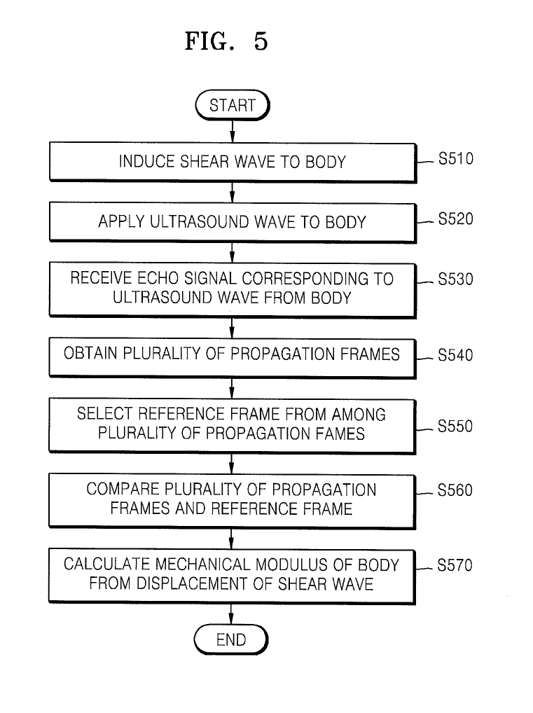

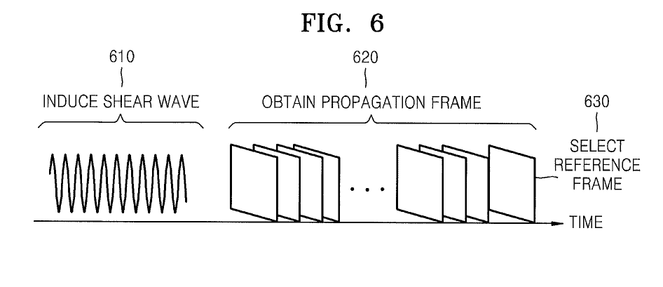

FIG. 5 is a flow chart illustrating a method of calculating a mechanical modulus of a tissue in a body by using a shear wave according to an embodiment of the present invention. FIG. 6 is a reference drawing illustrating induction of a shear wave and frame obtaining along time (i.e., with respect to a period of time).

Referring to FIGS. 5 and 6, the ultrasound probe 10 generates a shear wave in the body (operation S510). A shear wave may be induced 610 by applying an ultrasound signal with a predetermined frequency for a predetermined period of time to a focus area using some or all of the elements of a transducer. The ultrasound probe 10 inducing the shear wave may include a transducer as a single device. The transducer may be an HIFU transducer. Alternatively, the ultrasound probe 10 may include a plurality of transducers, and some or all of the transducers may be activated to apply an ultrasound signal for inducing the shear wave in the body. The ultrasound waves for inducing the shear wave may also cause physical changes in the body.

After the shear wave is induced, the ultrasound probe 10 applies an ultrasound signal to an area of interest 40 of the body (operation S520), and receives an echo signal of the shear wave from the body (operation S530). The ultrasound signal used for receiving the echo signal may have a smaller period (higher frequency) than the ultrasound signal used for generating the shear wave. That is, an intensity of the ultrasound signal for inducing the shear wave may be greater than an intensity of the ultrasound signal for obtaining the propagation frames. The ultrasound probe 10 converts the echo signal into an electrical signal and sends it to the shear wave processing apparatus 20.

The frame obtaining unit 210 of the shear wave processing apparatus 20 may perform beamforming on the electrical signal corresponding to the echo signal to obtain a plurality of propagation frames 620 (operation S540). A propagation frame refers to an ultrasound image of the area of interest 40, and shows the shear wave. The frame obtaining unit 210 may sequentially obtain the plurality of propagation frames in (over) a constant time interval, after the shear wave is induced in the area of interest. The frame obtaining unit 210 may perform beamforming on the echo signal according to the time when the transducers respectively apply the ultrasound waves, the time when the echo signal arrives at the transducers from the area of interest area 40, or a combination thereof.

The displacement calculating unit 220 may select a reference frame 630 from among a plurality of propagation frames (operation S550). For example, the displacement calculating unit 220 may select the last obtained propagation frame from among the plurality of the propagation frames as the reference frame, or a propagation frame obtained after the propagation frames pass through the area of interest 40.

In addition, the displacement calculating unit 220 may compare each of the plurality of propagation frames and the reference frame to calculate the displacement of the shear wave (operation 560). When comparing the propagation frames and the reference frame, a cross-correlation scheme may be applied.

Furthermore, the modulus calculating unit 230 calculates a mechanical modulus of a tissue in the area of interest 40 (operation S570). For example, the modulus calculating unit 230 calculates a travel velocity of the shear wave by using displacement components, which respectively correspond to coordinate axes and are included in the displacement of the shear wave. In addition, the shear modulus may be calculated by squaring the calculated travel velocity and multiplying the squared result by a density of the tissue. In addition to, or alternatively, the modulus calculating unit 230 may calculate a stiffness or viscosity of the tissue by using the displacement of the shear wave.

In order to obtain an image showing a displacement of a shear wave, a shear wave may be induced by applying an ultrasound signal with a predetermined frequency for a predetermined period of time to a focus area using some or all of the elements of a transducer. For example an ultrasound signal having a frequency of 5 MHz may be applied for 0.1 ms to a focus area of an exemplary model of a human tissue by using 36 elements from among 128 elements of a transducer. In addition, propagation frames may be obtained at a certain frame rate for the area of interest. Using the above example, propagation frames may be obtained at a frame rate of 7,800 frames/s for the area of interest of the exemplary model. As a comparative example, an ultrasound image of an area of interest may be selected as a reference frame before a shear wave is generated, and an image showing a displacement of the shear wave is obtained by comparing the reference frame and a plurality of propagation frames. In contrast, in an embodiment of the present invention, an ultrasound image may be selected as a second reference image after a shear wave is generated and the shear wave passes through the area of interest, and an image showing a displacement of the shear wave is obtained by comparing a plurality of propagation frames with the second reference frame.

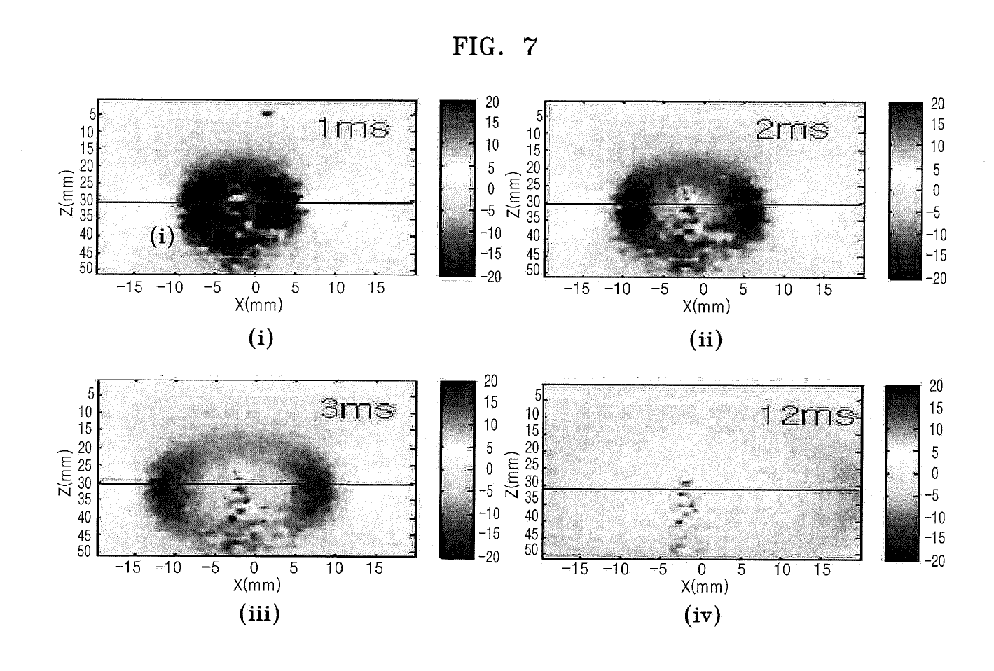

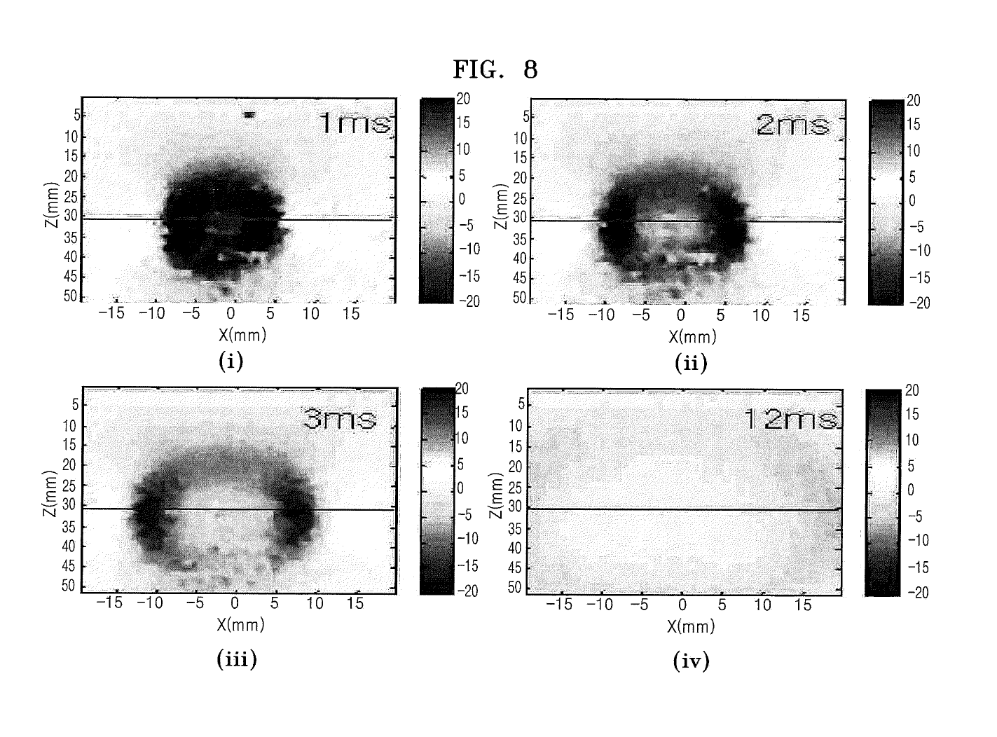

FIG. 7 illustrates a result of the comparative example of the shear wave displacement in time (with respect to a period of time), and FIG. 8 illustrates a result of an embodiment of the present invention showing the shear wave displacement in time (with respect to a period of time).

As shown in FIG. 7, when an ultrasound image of an area of interest is set as a reference frame before the shear wave is induced, and a shear wave travels away from the focus area within a phantom (i.e., a material or medium which mimics or simulates properties or characteristics of another material or medium such as a tissue or organ, for example) at a time after 1 ms, 2 ms, 3 ms, and 12 ms, and an afterimage remains around the focus area. The afterimage may cause miscalculation of a mechanical modulus of a tissue. In contrast, when a reference frame is selected from among propagation frames after a shear wave is induced, no afterimage remains around the focus area in an image showing a displacement of the shear wave, because the afterimage included in the reference frame is canceled out when the displacement of the shear wave is calculated.

An experiment has been performed on a phantom having a background shear modulus of 10 kPa and an inclusion with a diameter of 1 cm. In a comparative example, an ultrasound image of an area of interest before a shear wave is induced is selected as a reference frame, and a shear modulus is calculated by using a displacement of the shear wave after the displacement of the shear wave is calculated by comparing the reference frame and propagation frames. Then, an ultrasound image showing the shear modulus is created. In this experiment, a plurality of propagation frames have been obtained after the shear wave is induced. The last obtained propagation frame from among the plurality of propagation frames is selected as a reference frame. The reference frame and each of the propagation frames are compared to calculate the displacement of the shear wave. The shear modulus is calculated by using the displacement of the shear wave, and the ultrasound image showing the shear modulus is created.

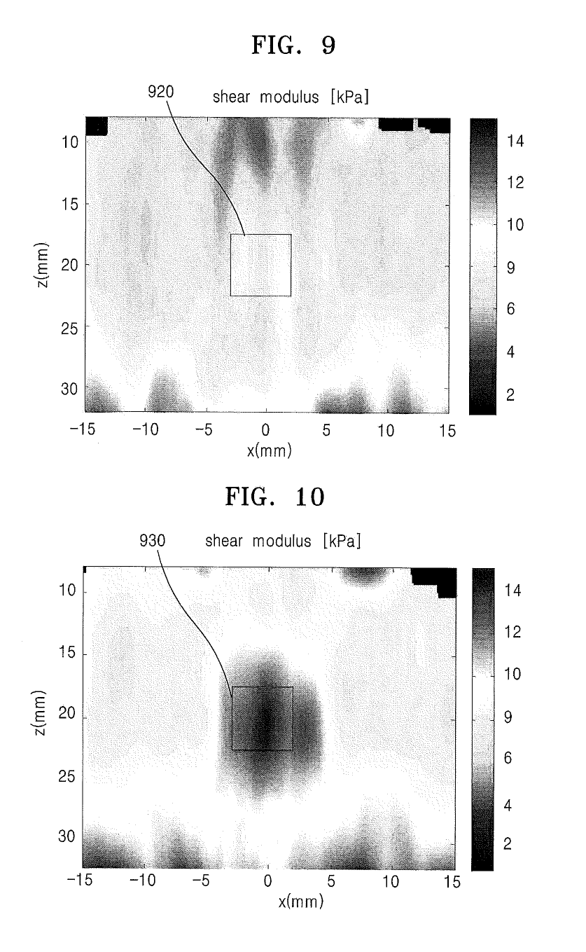

FIG. 9 shows an image showing the shear modulus according to the comparative example. FIG. 10 shows an image showing the shear modulus according to an embodiment of the present invention.

In the image according to the comparative example, with reference to reference numeral 920 in FIG. 9, no inclusion is present, while in the image according to the embodiment of the present invention, with reference to reference numeral 930 in FIG. 10, an inclusion is clearly present. That is, by selecting one of the ultrasound images as a reference image after a shear modulus is calculated, a mechanical modulus can be obtained.

In an embodiment, a mechanical modulus may be obtained by selecting one of the ultrasound images as a reference image after a shear modulus is calculated. However, the present invention is not limited thereto. A combination of ultrasound images after a shear wave is induced may be selected as a reference image, or a combination of ultrasound images after a shear wave is induced and ultrasound images before the shear wave is generated may be selected as a reference image. For example, an average image of ultrasound images after a shear wave is generated may be selected as a reference image, or an average image of ultrasound images after a shear wave is induced and ultrasound images before the shear wave is induced may be selected as a reference image. Alternatively, a frame whose signal to noise ratio (SNR) is the highest from among ultrasound images after an shear wave is induced may be selected as a reference frame, or an average image of ultrasound images whose SNR is equal to a threshold value or higher may be selected as a reference frame.

As described above, according to the one or more of the above embodiments of the present invention, a displacement of a shear wave can be calculated precisely by selecting a reference frame from among propagation frames after the shear wave is induced.

In addition, a mechanical modulus of a tissue can be calculated precisely because the displacement of the shear wave does not include information of physical changes of the tissue according to the induction of the shear wave.

In addition, other embodiments of the present invention can also be implemented through computer readable code/instructions in/on a medium, e.g., a non-transitory computer readable medium, to control at least one processing element to implement any above described embodiment. The medium can correspond to any medium/media permitting the storage and/or transmission of the computer readable code.

The computer readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), hardware devices that are specially configured to store and perform program instructions, such as read-only memory (ROM), random access memory (RAM), flash memory, and the like, and transmission media such as Internet transmission media. Thus, the medium may be such a defined and measurable structure including or carrying a signal or information, such as a device carrying a bitstream according to one or more embodiments of the present invention. The media may also be a distributed network, so that the computer readable code is stored/transferred and executed in a distributed fashion. Furthermore, the processing element could include a processor or a computer processor, and processing elements may be distributed and/or included in a single device. In addition, the computer-readable storage media may also be embodied in at least one application specific integrated circuit (ASIC) or Field Programmable Gate Array (FPGA).

The disclosure herein has described one or more embodiments in which a shear wave processing system and methods to calculate a displacement of a shear wave and mechanical modulus of a body may be used in medical applications to detect cancerous tissues or tumors, for the treating and diagnosing patients (e.g., humans, animals, and other lifeforms). However, the shear wave processing system and corresponding methods disclosed herein need not be limited to the medical field, and may be used in other fields, and may be used on an object in industrial applications to examine internal characteristics and structures of an object.

The apparatuses, systems, and methods according to the example embodiments disclosed herein may use one or more processors, which may include a microprocessor, central processing unit (CPU), digital signal processor (DSP), or application-specific integrated circuit (ASIC), as well as portions or combinations of these and other processing devices.

Each block of the flowchart illustrations may represent a unit, module, segment, or portion of code, which comprises one or more executable instructions for implementing the specified logical function(s). It should also be noted that in some alternative implementations, the functions noted in the blocks may occur out of the order. For example, two blocks shown in succession may in fact be executed substantially concurrently or the blocks may sometimes be executed in the reverse order, depending upon the functionality involved.

It should be understood that the exemplary embodiments described therein should be considered in a descriptive sense only and not for purposes of limitation. Descriptions of features or aspects within each embodiment should typically be considered as available for other similar features or aspects in other embodiments.

Although a few embodiments have been shown and described, it would be appreciated by those skilled in the art that changes may be made to these embodiments without departing from the principles and spirit of the invention, the scope of which is defined in the claims and their equivalents.

What is claimed is:

1. A method of calculating a displacement of a shear wave, the method comprising:
inducing a shear wave to a body;
obtaining a plurality of propagation frames including propagation information of the shear wave from an echo signal received from the body;
determining a reference frame from among the plurality of propagation frames; and
calculating a displacement of the shear wave based on the plurality of propagation frames and the reference frame.

2. The method according to claim 1, wherein the reference frame is a last obtained propagation frame from among the plurality of propagation frames.

3. The method according to claim 1, wherein the reference frame is a propagation frame obtained after the shear wave passes through an interest area of the body.

4. The method according to claim 1, wherein the displacement of the shear wave does not comprise information of physical changes of the body according to the induction of the shear wave.

5. The method according to claim 1, wherein the inducing the shear wave comprises applying an ultrasound signal to the body.

6. The method according to claim 5, wherein an intensity of the ultrasound signal for inducing the shear wave is greater than that for obtaining the propagation frames.

7. The method according to claim 1, wherein the calculating the displacement of the shear wave comprises applying a cross-correlation scheme to the propagation frames and the reference frame.

8. The method according to claim 1, further comprising displaying an image showing the displacement of the shear wave.

9. A non-transitory computer readable medium having recorded thereon a program to execute the method of claim 1 on a computer.

10. A method of calculating a mechanical modulus of a body by using a shear wave, the method comprising:
inducing a shear wave to a body;
obtaining a plurality of propagation frames including propagation information of the shear wave from an echo signal received from the body;
determining a reference frame from among the plurality of propagation frames;
calculating a displacement of the shear wave based on the plurality of propagation frames and the reference frame; and
calculating a mechanical modulus of the body from the displacement of the shear wave.

11. The method according to claim 10, wherein the mechanical modulus is at least one of a shear modulus, stiffness, and viscosity of the body.

12. The method according to claim 11, wherein the shear modulus is calculated from a velocity of the shear wave and a density of the body, and the velocity of the shear wave is calculated from the displacement of the shear wave.

13. The method according to claim 10, further comprising displaying an image showing the mechanical modulus.

14. A non-transitory computer readable medium having recorded thereon a program to execute the method of claim 9 on a computer.

15. An apparatus for processing a shear wave, comprising:
a frame obtaining unit to obtain a plurality of propagation frames including propagation information of a shear wave in a body; and
a displacement calculating unit to select a reference frame from among the plurality of propagation frames, and to compare the reference frame and the plurality of propagation frames to calculate a displacement of the shear wave.

16. The apparatus according to claim 15, wherein the displacement calculating unit selects a last obtained propagation frame from among the plurality of propagation frames as the reference frame.

17. The apparatus according to claim 15, wherein the displacement calculating unit selects a propagation frame obtained after the shear wave passes through an area of interest in the body as the reference frame.

18. The apparatus according to claim 15, wherein the displacement of the shear wave does not comprise information of physical changes of the body according to the induction of the shear wave.

19. A system for processing a shear wave, the system comprising:
an ultrasound probe to apply an ultrasound signal to a body; and
an apparatus for processing a shear wave, the apparatus comprising:
a frame obtaining unit to obtain a plurality of propagation frames including propagation information of a shear wave in a body; and
a displacement calculating unit to select a reference frame from among the plurality of propagation frames, and to compare the reference frame and the plurality of propagation frames to calculate a displacement of the shear wave.

20. The system according to claim 19, wherein the ultrasound probe induces the shear wave by applying the ultrasound signal to the body.

21. The system according to claim 19, wherein the ultrasound probe applies the ultrasound signal to the body, and receives an echo signal corresponding to the plurality of propagation frames from the body.

22. A method of determining characteristics of an object, the method comprising:
generating a shear wave in an object;
applying, after the shear wave is generated, an ultrasound signal to the object and obtaining a plurality of propagation frames from an echo signal corresponding to the ultrasound signal;
selecting, after the shear wave is generated, a reference frame from among the plurality of propagation frames; and
determining a displacement of the shear wave based on a relationship between the plurality of propagation frames and the reference frame.

23. The method according to claim 22, further comprising:
determining a shear modulus of the object using a travel velocity and the calculated displacement of the shear wave including displacement components corresponding to a plurality of axes.

24. The method according to claim 22, wherein the ultrasound signal applied to the object after the shear wave is generated has a smaller period than an ultrasound signal used to generate the shear wave.

25. The method according to claim 22, wherein after the shear wave is generated, characteristics of a plurality of propagation frames are combined to obtain a reference frame.

26. The method according to claim 22, wherein a propagation frame having a highest signal to noise ratio among the plurality of propagation frames is selected as the reference frame.

27. A non-transitory computer readable medium having recorded thereon a program to execute the method of claim 22 on a computer.
